(19) United States
(12) Patent Application Publication
(10) Pub. No.: US 2006/0281104 A1
(43) Pub. Date: Dec. 14, 2006

(54) LEUCO DYE PARTICLES AND USES THEREOF

(76) Inventor: STEPHEN C. MACEVICZ, Cupertino, CA (US)

Correspondence Address: STEPHEN C. MACEVICZ, 21890 RUCKER DRIVE, CUPERTINO, CA, 95014, US

(21) Appl. No.: 11/381,569

(22) Filed: May 4, 2006

Related U.S. Application Data
(60) Provisional application No. 60/689,805, filed on Jun. 13, 2005.

(51) Int. Cl. C12Q 1/68; G01N 33/551

(52) U.S. Cl. 435/6; 977/924; 436/524

(57) ABSTRACT

The invention provides methods and compositions for detecting the presence of, or for measuring amounts of, molecular targets, particularly molecular complexes comprising two or more proteins, such as receptor complexes of cell surface membranes. In one aspect of the invention, reagent pairs are provided that comprise a reactive species generator that specifically binds to a first region of a molecular target and one or more signaling reagents that each specifically bind to one or more second regions that do not overlap with the first region. In one aspect, each signaling reagent comprises a binding compound specific for a molecular target, such as a component of the molecular complex, and attached to the binding compound, a nanoparticle having one or more leuco dyes attached. In one embodiment, such nanoparticles are releasable bound to the binding compound, so that the nanoparticles may be released and isolated for analysis in a controlled manner as part of an assay protocol. Reactive species generators can be induced to generate a reactive species that, within a characteristic proximity, is capable of reacting with leuco dyes so that they are converted into fluorescent labels.

This application claims priority from U.S. provisional patent application Ser. No. 60/689,805 filed 13 Jun. 2005, which is incorporated herein by reference.

### FIELD OF THE INVENTION

The present invention relates generally to assays for biomolecules, and more particularly to the detection and measurement of complexes of biomolecules.

### BACKGROUND OF THE INVENTION

The formation and disassociation of molecular complexes is a pervasive biological phenomenon that is crucial to regulatory processess in living organisms. For example, the interations of several cell surface membrane components play crucial roles in transmitting extracellular signals to a cell in normal physiology, and in disease conditions. Many types of cell surface receptors undergo dimerization, oligomerization, or clustering in connection with the transduction of an extracellular event, such as ligand-receptor binding, into a cellular response, such as proliferation, increased or decreased gene expression, or the like, e.g. George et al. Nature Reviews Drug Discovery, 1:808-820 (2002); Mellado et al. Ann. Rev. Immunol., 19, 397-421 (2000); Schlessinger, Cell, 103: 211-225 (2000); Yarden, Eur. J. Cancer, 37: S3-S8 (2001). The role of such signal transduction events in diseases, such as cancer, has been the object of intense research and has led to the development of several new drugs and drug candidates, e.g. Herbst and Shin. Cancer, 94: 1593-1611 (2002): Yarden and Sliwkowski, Nature Reviews Molecular Cell Biology, 2: 127-137 (2001); McCormick, Trends in Cell Biology, 9: 53-56 (1999); Blume-Jensen and Hunter, Nature, 411: 355-365 (2001); Baselga, Cancer Cell. 2: 93-95 (2002); Agus et al, Cancer cell, 2,: 127-137 (2002), Koll et al, International patent publications WO2004/008099.

A wide variety of techniques have been used cellular protein-protein interactions and complexes, including immunoprecipitation, chemical cross-linking, bioluminescene resonance energy transfer (BRET), fluorescence resonance energy transfer (FRET), and the like, e.g. Price et al. Methods in Molecular biology, 218: 255-267 (2003); Sorkin et al, Curr. Biol 10: 1395-1398 (2000); McVey et al, J. Biol. Chem., 17: 14092-14099 (2001): Salim et al, J. Biol. Chem., 277: 15482-15485 (2002): Angers et al, Proc. Natl. Acad. Sci., 97: 3684-3689-3689 (2000); Stagljar, STKE Science 2003, pe56 (2003). Unfortunately, such techniques are frequently difficult to apply, especially in a clinical setting , require relatively large sample sizes, and generally lack sufficient sensitivity to provide an accurate picture of complex molecular interactions, such as those related to signaling pathways. Techniques based on releasable molecular tags have been proposed for detecting multiple analytes, including molecular complexes; however, such approaches require specialized separation equipment for implementation, and the paucity of performance data related to their applications makes it difficult to evaluation the utility of such approaches for detecting molecular complexes, e.g. Giese, Trends in Anal. Chem., 2: 165-167 (1983); Giese, U.S. Pat. No. 4,650,750 and 4,709,016; Tian et al, Nucleic Acids Research, 32(16): e126 (2004); Ricco et al, Biochem. Soc. Trans., 30(2): 73-78 (2002); and Chan-Hui et al, International patent publication WO 2004/011900.

In view of the above, the availability of a convenient, sensitive, and cost effective technique for simultaneously detecting or measuring multiple analytes or one or more molecular complexes, particularly those in signaling pathways, would advance many fields where such measurements are becoming increasingly important, including life science research, medical research and diagnostics, drug discovery, and the like.

### SUMMARY OF THE INVENTION

The invention provides methods and compositions for detecting the presence of, or for measuring amounts of, molecular targets, particularly molecular complexes comprising two or more proteins, such as receptor complexes of cell surface membranes. In one aspect of the invention, reagent pairs are provided that comprise a reactive species generator that specifically binds to a first region of a molecular target and one or more signaling reagents that each specifically bind to one or more second regions that do not overlap with the first region. in another aspect, reagent pairs are provided that comprise a reactive species generator that specifically binds to at least one component of a molecular complex and one or more signaling reagents that specifically bind to one or more components of the molecular complex, at least one of which is different from the component to which the reactive species generator is attached. In one aspect, each signaling reagent comprises a binding compound specific for a molecular target, such as a component of the molecular complex, and attached to the binding a compound, a nanoparticle having one or more leuco dyes attached. In one embodiment, the nanoparticles are releasably bound to the binding compund, so that the nanoparticles may be released and isolated for analysis in a controlled manner as part of an assay protocol. Reactive species generators can be induced to generate a reactive species that, within a characteristic proximity, is capable of reacting with leuco dyes so that they are converted into fluorescent labels.

In one aspect, the invention provides compositions comprising a plurality of distinct nanoparticles each having (i) a binding compound and one or more leuco dyes attached and (ii) an indicator compound, such that binding compounds of different nanoparticles of the plurality have different specificities and indicator compounds of different nanoparticles generate different characteristic optical signals.

In accordance with one aspect of the invention, signaling reagents and reactive species generators are combined with a sample containing a molecular complex so that each reagent can specifically bind to its respective target component, if present in the sample. Whenever signaling reagents are bound to a molecular complex within the characteristic proximity of a reactive species generator, leuco dyes are converted into fluorescent labels. Nanoparticles carrying the fluorescent labels are then released and detected to indicate the presence or amount of the molecular complex.

In one aspect, the method of the invention is carried out with the following steps: (i) providing a binding compound specific for the first component, the binding compound having one or more particles attached, wherein each particle has one or more leuco dyes attached; (ii) providing a cleaving probe specific for the second component, the cleaving probe having a reactive species generator capable of generating a reactive species within a effective proximity, and the reactive species being capable of reacting with a leuco dye to form a signal generating moieyt; (iii) combining with the sample in an assay mixture the binding compound and the cleaving probe so that the binding compound and cleaving probe specifically bind to the first and second components, respectively, and so that whenever the first component and the second component are in a molecular complex, leuco dyes of a t least one particle are within the effective proximity of the reactive species generator and are converted into signal generating moieties by the reactive species; and (iv) detecting a signal from the signal generating moieties and relating the signal to the presence or amount of the molecular complex in the sample.

In another aspect, the invention provides a method of measuring relative phosphorylation levels of one or more proteins. In one embodiment such method is carried out with the following steps: (i) providing a first binding compound specific for a phosphorylation site of a protein in a phosphorylated state, the first binding compound having one or more particles attached, wherein each particle has at least one first indicator molecule and one or more leuco dyes attached; (ii) providing a second binding compound specific for the phosphorylation site of the protein in an unphosphorylated state, the second binding compound having more particles attached, wherein each particle has at least one second indicator molecule and one or more leuco dyes attached; (iii) providing a cleaving probe specific for an antigenic determinant different from the phosphorylation site, the cleaving probe having a reactive species generator capable of generating a reactive species a within an effective proximity, and the reactive species being capable of reacting with a leuco dye to form a signal generating moiety; (vi) combining with the sample in an assay mixture the first and second binding compounds and the cleaving probe so that the first and second binding compounds and cleaving probe specifically bind to their respective antigentic determininants; (v) activating the reactive species generator so that reactive speciesare generated to produce signal generating moieties; and (v) detecting signals from the first and second indicator molecules and the signal generating moieties and relating the signals to the presence or relative amount of the protein in a phosphorylated and unphosphorylated state in the sample.

In another aspect, the invention provides compositions comprising a plurality of nanoparticles each having a surface and each having attached to such surface (i) leuco dyes capable of being converted into detection moieties by a reactive species, the detection moieties being capable of generating a first optical signal and (ii) one or more binding compounds each having a specificity for an antigen, such that the specificity of the one or more binding compounds on the same nanoparticle is for the same antigen, and such that the specificity of the one or more binding compounds on each different nanoparticle of the plurality is for a different antigen. In still another aspect, the above nanoparticles each have attached to their surface an indicator molecule. In one embodiment, such indicator molecules are non-reactive with at least one reactive species. In another embodiment, each different indicator molecule is capable of producing a different spectrally resolvable fluorescent signal.

In another aspect, the invention provides compositions comprising a plurality of nanoparticles each having a surface and each having attached to such surface (i) leuco dyes capable of being converted into detection moieties by a reactive species, the detection moieties being capable of generating a first optical signal and (ii) at least one reactive group capable of reacting with a reciprocal moiety to form a stable linkage, such as with a binding compound. In still another aspect, the above nanoparticles each have attached to their surface an indicator molecule. In one embodiment, such indicator molecules are non-reactive with at least one reactive species. In another embodiment, each nanoparticle of the plurality has a different indicator molecule. In still another embodiment, each different indicator molecule is capable of producing a different spectrally resolvable fluorescent signal.

In one aspect, each of the above-mentioned pluralities is in the range of from 2 to 10; in another aspect, each of such pluralities is in the range of from 2 to 6; and in still another aspect, each of such pluralites is in the range of from 2 to 4.

### BRIEF DESCRIPTION OF THE DRAWINGS

FIGS. 1A-1F illustrate diagrammatically an embodiment of the method of the invention using a single leuco dye for measuring the presence of intracellular complexes.

FIGS. 2A-2E illustrate diagrammatically an embodiment of the method of the invention using multiple leuco dyes for measuring the presence of molecular complexes.

FIGS 3A-3D illustrate the use of photosensitizer-impregnated microbeads in an embodiment of the invention.

FIGS 4A-4B illustrate the use of compounds of the invention to measure relative phosphorylation states of one or more proteins.

### DEFINITIONS

“Antibody” means an immunoglobulin that specifically binds to, and is thereby defined as complementary with, a particular spatial and polar organization of another molecule. The antibody can be monoclonal or polyclonal and can be prepared by techniques that are well known in tha art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and iso types, such as IgA, IgD, IgE, IgGI, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding afinity for a particular polypeptide is maintained. Guidance in the production and selection of antibodies for use in immunoassays, including such assays employing releasable molecular tag (as described below) can be found in readily available texts and manuals, e.g. Harlow and Lane, antibodies: A Laboratory Manual (Cold Spring Harbor Laboratory Press, New York, 1988); Howard and Bethell, Basic Methods in Antibody Production and Characterization (CRC Press, 2001); Wild, editor, The Immunoassay Handbook (Stockton Press, New York, 1994), and the like.

“Antibody binding composition” means a molecule or a complex of molecules that comprises one or more antibodies, or fragments thereof, and drives its binding specificity from such antibody or antibody fragment. Antibody binding compositions include, but are not limited to, (i) antibody pairs in which a first antibody binds specifically to a target molecule and a second antibody binds specifically to a constant region of the first antihbody; a biotinylated antibody that binds specifically to a target molecule and a streptavidin protein, which protein is derivatized with moieties such as molecular tags or photosensitizers, or the like, via a biotin moiety; (ii) antibodies specific for a target molecule and conjugated to a polymer, such as dextran, which, in turn, is derivatized with moieties such as molecular tags or photosensitizerzs, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as molecular tags or photosensitizers, or polymers containg the latter.

“Antigentic determinant,” or “epitope” means a site or the surface of a molecule, usually a protein, to which a single antibody molecule binds; generally a protein has several or many different antigenic determinants and reacts with antibodies of many different specificities. A preferred antigenic determinant is a phosphorylation site of a protein.

“Binding moiety” means any molecule to which molecular tags can be directly or indirectly attached that is capable of specifically binding to an analyte. Binding moieties include, but are not limited to, antibodies, antibody binding compositions, peptides, proteins, nucleic acids, and organic molecules having a molecular weight of up to 1000 daltons and consisting of atoms selected from the group consisting of hydrogen, carbon, oxygen, nitrogen, sulfur, and phosphorus. Preferably, binding moieties are antibodies or antibody binding compositions.

“Complex” as used herein means an assemblage or aggregate of molecules in direct or indirect contact with one another. In one aspect, “contact,” or more particularly, “direct contact” in reference to a complex of molecules, or in reference to specificity or specific binding, means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its xomponent molecules. As used herein, “complex” usually refers to a stable aggregate of two or more proteins, and is equivalently referred to as a “protein-protein complex.” Most typically, a “complex” refers to a stable aggregate of two proteins.

“Dimer” in reference to cell surface membrane receptors means a complex of two or more membrane-bound receptor proteins that may be the same or diferent. Dimers of identical receptors are referred to as “homodimers” and dimers of different receptors are referred to as “heterodimers.” Dimers usually consist of two receptors in contact with one another. Dimers may be created in a cell surface membrane by passive processes, such as Van der Waal interactions, and the like, as described above in the definition of “complex,” or dimers may be created by active processes, such as by ligand-induced dimerization, covalent linkages, interaction with intracellular components, or the like, e.g. Schlessinger, Cell, 103: 211-225 (2000). As used herein, the term “dimer” is understood to refer to “cell surface membrane receptor dimer,” unless understood otherwise from the context.

“ErbB receptor” or “Her receptor” is a receptor protein tyrosine kinase which belongs to the ErbB receptor family and includes EGFR (“Her1”), ErbB2 (“Her2”), ErbB3 (“Her3”) and ErbB4 (“Her4”) receptors. The ErbB receptor generally comprises an extracellular domain, which may bind an ErbB ligand; a lipophilic transmembrane domain; a conserved intracellular tyrosine kinase domain; and a carboxyl-terminal signaling domain harboring several tyrosine residues which can be phosphorylated. The ErbB receptor may be a native sequence ErbB receptor or an amino acid sequence variant thereof. Preferably the ErbB receptor is native sequence human ErbB receptor.

The terms “ErbB1”, “epidermal growth factor receptor” and “EGFR” and “Her1” are used interchangeably herein and refer to native sequence EGFR as disclosed, for example, in Carpenter et al. Ann. Rev. Biochem. 56:881-914 (1987), including ariants thereof (e.g. a deletion mutan EGFR as in Humphrey et al. PNAS (USA) 87:4207-42211 (1990)), erbB1 refers to the gene encoding the EGFR protein product. Examples of antibodies which bind to EGFR include MAb 579 (ATCC CRL RB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.).

“Her2”, “ErbB2” “c-Erb-B2” are used interchangeably. Unless indicated otherwise, the terms “ErbB2” “c-Erb-B2” and “Her2” when used herein refer to the human protein. The human ErbB2 gene and ErbB2 protein are, for example, described in Semba et al., PNAS (USA) 82:6497-650 (1985) and Yamamoto et al. Nature 319:230-234 (1986) (Genebank accession number X03363). Examples of antibodies that specifically bind to Her2 are disclosed in U.S. Pat. Nos. 5,677,171; 5,772,997; Fendly et al. Cancer Res., 50: 1550-1558 (1990); and the like.

“ErbB3” and “Her3” refer to the receptor polypeptide as disclosed, for example, in U.S. Pat. Nos. 5,183,884 and 5,480,968 as well as Kraus et al. PNAS (USA) 86:9193-9197 (1989), including variants thereof. Examples of antibodies which bind Her3 are described in U.S. Pat. No. 5,968,511, e.g. the 8B8 antibody (ATCC HB 12070).

The terms “ErbB4” and “Her4” herein refer to the receptor polypeptide as disclosed, for example, in Plowman et al., Proc. Natl. Acad. Sci. USA, 90:1746-1750 (1993); and Plowman et al., Nature, 366:473-475 (1993). Antibodies to Her4 are disclosed in U.S. patent SSSSSSSS

“Isolated” in reference to a polypeptide or protein means substantially separated from the components of its natural environment. Preferably, an isolated polypeptide or protein is a composition that consists of at least eighty percent of the polypeptide or protein identified by sequence on a weight basis as compared to components of its natural environment; more preferably, such composition consists of at least ninety-five percent of the polypeptide or protein identified by sequence on a weight basis as compared to components of its natural environment; and still more preferably, such composition consists of at least ninety-five percent of the polypeptide or protein identified by sequence on a weight basis as compared to components of its natural environment. Most preferably, an isolated polypeptide or protein is a homogeneous composition that can be resolved as a single spot after conventional separation by two-dimensional gel electrophoresis based on molecular weight and isoelectric point. Protocols for such analysis by conventional two-dimentsional gel electrophoresis are well known to one of oridinary skill in the art, e.g. Hames and Rickwood, Editors, Gel Electrophoresis of Proteins: A Practical Approach (IRL Press, Oxford, 1981); Scopes, Protein Purification (Springer-Verlag, New York, 1982); Rabilloud, Editor, Proteome Research: Two-Dimensional Gel Electrophoresis and Identification Methods (Springer-Verlag, Berlin, 2000).

“Kit” refers to any delivery system for delivering materials or reagents for carrying out a method of the invention. In the context of reation assays, such delivery systems include systems that allow for the storage, transport, or delivery of reaction reagents (e.g., probes, enzymes, etc. in the appropriate containers) and/or supporting materals (e.g., buffers, written instructions for performing the assay etc.) from one location to another. For example, kits include one or more enclosures (e.g., boxes) containing the relevant reaction reagents and/or supporting materials. such contents may be delivered to the intended recipient together or separately. For example, a first container may contain an enzyme for use in an assay, while a second container contains probes.

“Leuco Dye” means any compound whose optical characteristics may be changed by reaction with a reactive species. In one aspect, optical characteristics that are changed are fluorescent characteristic; in particular, a leuco dye may change from a substantially non-fluorescent compound to a fluorescent compound by reacting with a reactive species. In another aspect, a leuco dye is oxidized by a reactive species to form a product with altered optical characteristics; in particular, in such aspect, a leuco dye is oxidized to change it from a substantially non-fluorescent compound to a fluorescent compound. In another aspect, a leuco dye is a compound that is substantially non-fluorescent, but that may produce a fluorescent product upon reaction with singlet oxygen, or equivalent oxidant. A wide variety of leuco dyes are disclosed in the following references: Muthyala, editor, Chemistry and Applications of Leuco Dyes (Plenum Press, New York, 1997); and Haugland, Handbook of Fluorescent Probes and Research Products, Ninth Edition (Molecular Probes, Eugene, Oreg., 2002).

“Polypeptide” refers to a class of compounds composed of amino acid residues chemically bonded together by amide linkages with elimination of watr between the carboxy group of one amino acid and the amino group of another amino acid. A polypeptide is a polymer of amino acid residues, which may contain a large number of such residues. Peptides are similar to polypeptides, except that, generally, they are comprised of a lesser number of amino acids. Peptides are sometimes referred to as oligopeptides. There is no clear-cut distinction between polypeptides and peptides. For convenience, in this disclosure and claims, the term “polypeptide” will be used to refer generally to peptides and polypeptides. The amino acid residues may be natural or synthetic.

“Protein” refers to a polypeptide, usually synthesized by a biological cell, folded into a defined three-dimensional structure. Proteins are generally from about 5,000 to about 5,000,000 or more in molecular weight, more usually from about 5,000 to about 1,000,000 molecular weight, and may include postranslational modifications, such acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinsoitol, cross-linking, cyclization, disulfide bond formation, famesylation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, phosphorylation, prenylation, racemization, selenoylation, sulfation, and ubiquitination, e.g. Wold, F., Post-translational Protein Modifications: Perspectives and Prospects, pgs. 1-12 in Post-translational Covalent Modification of Proteins, B.C. Johnson, Ed., Academic Press, New York, 1983. Proteins include, by way of illustration and not limitation, cytokines or interleukns, enzymes such as, e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycans, and the like.

“Reference sample” means one or more cell or tissue samples that are representative of a normal or non-diseased state to which measurements on patient samples are compared to determine whether a receptor complex is present in excess or is present in reduced amount in the patient sample. The nature of the reference sample is a matter of design choice for a particular assay and may be derived or determined from normal tissue of the patient him- or herself, or from tissues from a population of healthy individuals. preferably, values relating to amounts of receptor complexes on reference samples are obtained under essentially identical experimental conditions as corresponding values for patient samples being tested. Reference samples may be from the same kind of tissue as that the patient sample, or it may be from different tissue types, and the population from which reference samples are obtained may be selected for characteristics that match those of the patient, such as age, sex race, and the like. Typically, in assays of the invention, amounts of receptor complexes on patient samples are compared to corresponding values of reference samples that have been previously tabulated and are provided as average ranges, average values with standard deviations, or like representations.

“Receptor complex” means a complex that comprises a dimer of cell surface membrane receptors. Receptor complexes may include one or more intracellular proteins, such as adaptor proteins, that form links in the various signaling pathways. Exemplary intracellular proteins that may be part of a receptor complex includes, but is not limit to, PI3K proteins, Grb2 proteins, Grb7 proteins, Shc proteins, and Sos proteins, Src proteins, Cb1 proteins, PLCγ proteins, Shp2 proteins, GAP proteins, Nck proteins, Bav proteins, and Crk proteins.

“Receptor tyrosine kinase, ” or “RTK,” means a human receptor protein having intracellular kinase activity and being selected from the RTK family of proteins described in Schlessinger, Cell, 103: 211-225 (2000); and Blume-Jensen and Hunter (cited above). “Receptor tyrosine kinase dimer” means a complex in a cell surface membrane comprising two receptor tyrosine kinase proteins. In some aspects, a receptor tyrosine kinase dimer may comprise two covalently linked receptor tyrosine kinase proteins.

“Sample” or “tissue sample” or “patient sample” or “patient cell or tissue sample” or “specimen” each means a collection of similar cells obtained from a tissue of a subject or patient. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; or cells from any time in gestation or development of the subject. The tissue sample may contain compounds which are not naturally intermixed with the tissue in nature such as preservatives, anticoagulants, buffers, fixatives, nutrients, antibiotics, or the like. In one aspect of the invention, tissue samples or patient samples are fixed, particularly conventional formalin-fixed paraffin-embedded samples. Such samples are typically used in an assay for receptor complexes in the form of thin sections, e.g. 3-10 μm thick, of fixed tissue mounted on a microscope slide, or equivalent surface. Such samples also typically undergo a conventional re-hydration procedure, and optionally, and antigen retrieval procedure as part of, or preliminary to , assay measurements.

“SHC” (standing for “Src hmology 2/α-collagen-related”) means any one of a family of adaptor proteins (66, 52, and 46 kDalton) in RTK signaling pathways substantially identical to those described in Pelicci et al, Cell, 70: 93-104 (1992). In one aspect, SHC means the human versions of such adaptor proteins.

“Signaling pathway” or “signal transduction pathway” means a series of molecular events usually beginning with the interaction of cell surface receptor with an extracellular ligand or with the binding of an intracellular molecule to a phosphorylated site of a cell surface receptor that triggers a series of molecular interactions, wherein the series of molecular interactions results in a regulation of gene expression in the nucleus of a cell. “Ras-MAPK pathway” means a signaling pathway that includes the phosphorylation of a MAPK protein subsequent to the formation of a Ras-GTP complex. “PI3K-Akt pathway” means a signaling pathway that includes the phosphorylation of an Akt protein by a PI3K protein.

“Specific” or “specificity” in reference to the binding of one molecule to another molecule, such as a binding compound, or probe, for a target analyte or complex, means the recognition, contact, and formation of a stable complex between the probe and target, together with substantially less recognition, contact, or complex formation of the probe with other molecules. In one aspect, “specific” in reference to the binding of a firs molecule to a second molecule means that to the extent the first molecule recognizes and forms a complex with another molecules in a reaction or sample, it forms the largest number of the complexes with the second molecule. In one aspect, this largest number is at least fifty percent of all such complexes form by the first molecule. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like.

“Spectrally resolvable” in reference to a plurality of fluorescent labels means that the fluorescent emission bands of the labels are sufficiently distinct, i.e. sufficiently non-overlapping, that molecular tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21-76, in Flow Cytometry: Instrumentation and Data Analysis (Academic Press, New York, 1985).

“Substantially identical” in reference to proteins or amino acid sequences of proteins in a family of related proteins that are being compared means either lat one protein has an amino acid sequence that is at least fifty percent identical to the other protein or that one protein is an isoform or splice variant of the same gene as the other protein. In one aspect, substantially identical means one protein, or amino acid sequence thereof, is at least eighty percent identical to the other protein, or amino acid sequence thereof.

“VEGF receptor” or “VEGFR” as used herein refers to a cellular receptor for vascular endothelial growth factor (VEGF), ordinarily a cell-surface receptor found on vascular endothelial cells, as well as variants thereof which retain the ability to bind human VEGF. VEGF receptors include VEGFR1 (also known as Flt1), VEGFR2 (also know as Flk1 or KDR), and VEGFR3(also known as Flt4). These receptors are described in DeVries et al., Science 255:989 (1992); Shibuya et al., Oncogene 5:519 (1990); Matthews et al., Proc. Nat. Acad. Sci. 88:9026 (1991); Terman et al., Oncogene 6:1677 (1991); Terman et al., Biochem. Biophys. Res. Commun. 187:1579 (1992). Dimers of VEGF receptors are described in shibuya. Cell Structure and Function, 26: 25-35 (2001); and Ferrara et al, Nature Medicine, 9: 669-676 (2003); and include VEGFR1 homodimers, VEGFR2 homodimers, VEGFR1-VEGFR2 heterodimers, and VEGFR2-VEGFR3 heterodimers.

### DETAILED DESCRIPTION OF THE INVENTION

In one aspect, the invention provides methods and compositions for detecting or measuring molecular targets, such as molecular compleses, particularly in samples of human tissues. In another aspect, an isolatable particle is used to record molecular events, such as the presence or formation of a molecular complex. Such a particle is “isolatable” in the sense that it may be removed or isolated from an assay reaction mixture for detection, or when removed as part of a population of particles, for detection and enumeration. In one form, such methods for determining the presence or amount of a molecular complex in a sample (where the molecular complex comprising at least a first component and a second component) by the following steps: (a) providing a binding compound specific for the first component, the binding compound having one or more particles attached, wherein each particle has one or more leuco dyes attached; (b) providing a cleaving probe specific for the second component, the cleaving probe having a reactive species generator capable of generating areactive species within an efective proximity, and thee reactive species being capabl of reacting with a leuco dye to form a signal generating moiety; (c) combining with the sample in an assay mixture the binding compound and the cleaving probe so that the binding compound and cleaving probe specifically bind to the first and second components, respectively, and so that whenever the first component and the second component are in a molecular complex, leuco dyes of at least one particle are within the effective proximity of the reactive species generator and are converted into signal generating moieties by the reactive species; and (d) detecting a signal from the signal generating moieties and relating the signal to the presence or amount of the molecular complex in the sample. In one aspect, each of said particles comprises an indicator compound that is capable of generating an indicator signal that is substantially unaffected by said reactive species. In another aspect, said particles are releasablely attached to said binding compounds. In another aspect, the method further including the step of releasing said particles. In another aspect, said released particles are isolated from said assay mixture and wherein said step of detecting includes detecting for each isolated particle said signal from said signal generating moieties and an indicator signal from said incicatior compound. In another aspect, the method further includes a step of computing for each of said isolated particles a signal ratio from said signal from said signal generating moieties and said indicator signal and relating said amount of said molecular complex to a number of particles having a signal ratio of a predetermined magnitude. In another aspect, said releasable particles are magnetic nanoparticles and werein after said step of releasing, magnetically isolating the magnetic nanoparticles from said assay mixture. In another aspect, said step of detecting includes detecting for each isolated nanoparticle said signal from said signal generating moieties and and indicator signal from said indicator compound. In another aspect, the methos further includes the step of computing for each of said isolated nanoparticles a signal ratio from said signal from said signal generating moieties and said indicator signal and relating said amount of said molecular complex to a number of nanoparticles having a signal ratio of a preetermined magnitude.

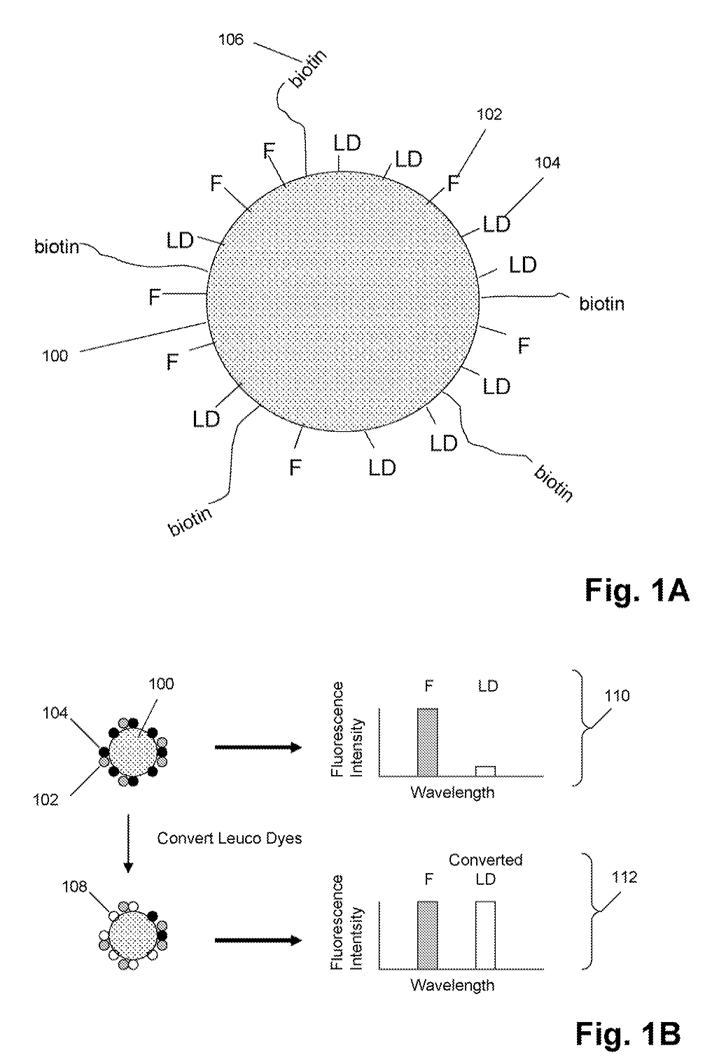

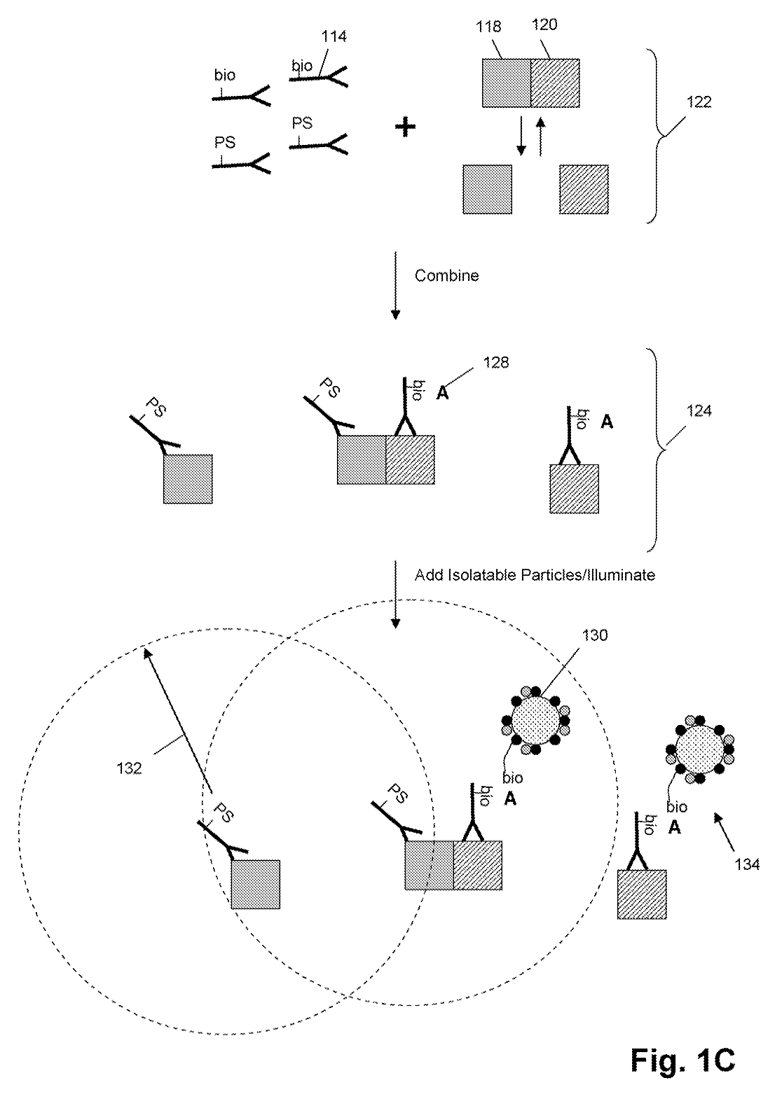

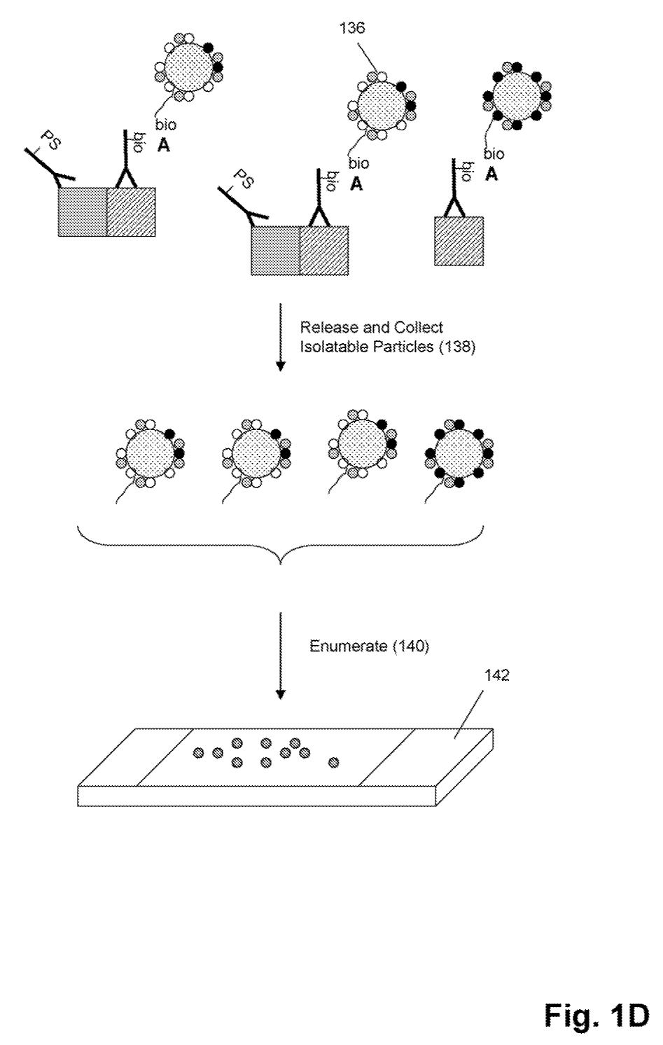

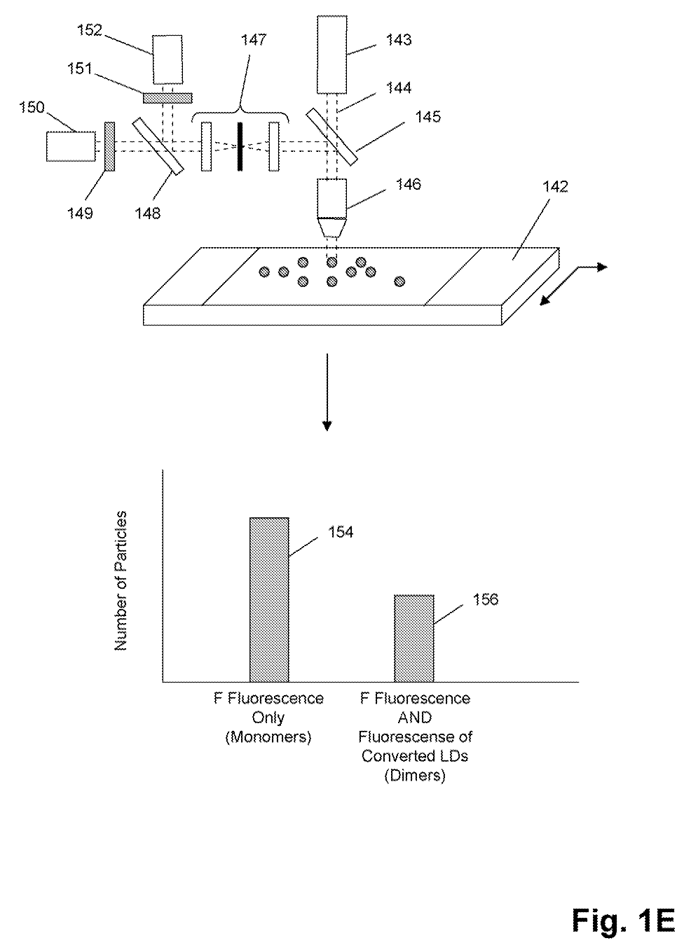

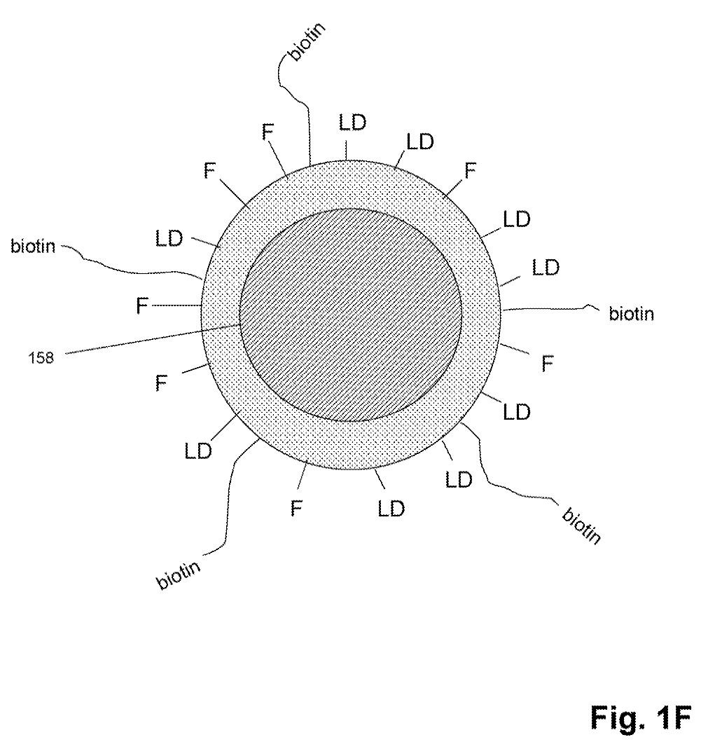

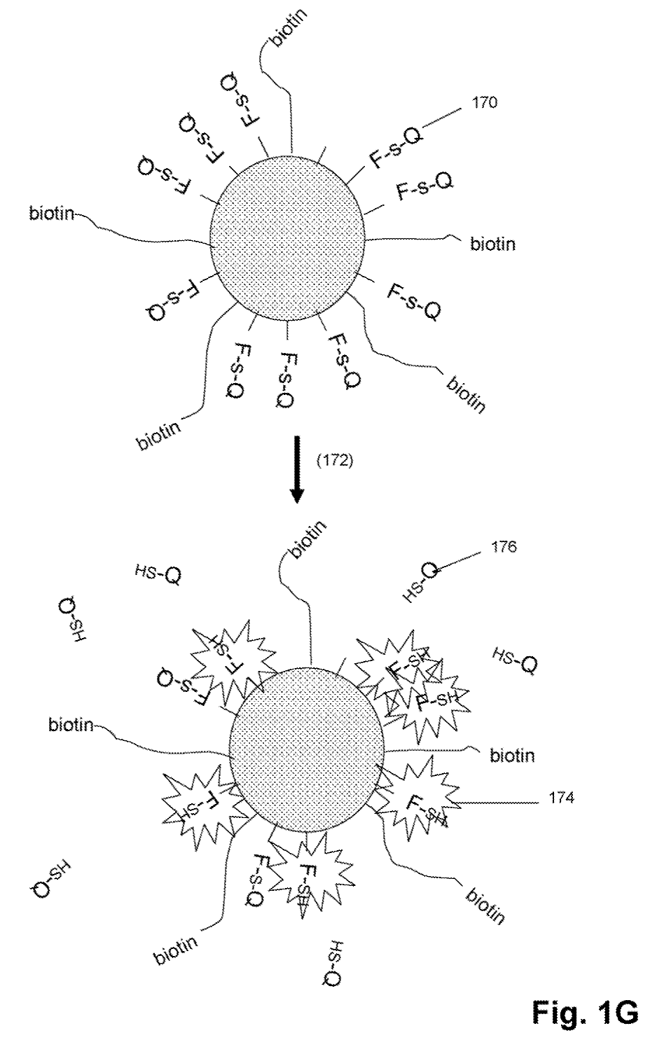

FIG. 1A shows diagrammatically an example of an isolatable particle. Particle (100) may vary widely in composition, size, and shape, as it serves as a platform for carrying various indicator molecules and other moieties for facilitating its participation in assay reactions, isolation, and detection. The size of particle (100) may vary between a few nanometers, e.g. about 10 nm, to a few micrometers, e.g. about 1-2 μm, with the proviso that functionally it must be large enough to carry a detectable amount of indicator molecules, coverted leuco dyes, or the like, and it must be small enough so that it does not interfere with the molecular complexes, or other molecular targets, it is being used to detect. In one aspect, particle (100 ) is a nanoparticle; that is, it has a size in the range of a few nanometers, e.g. 10 nm, to several hundred nanometers, e.g. 300 nm; or in the range of from 20 nm to 300 nm; or in the range of from 20 nm to 200 nm; or in the range from 30 nm to 150 nm; or in the range from 50 mn to 150 nm; or in the range from 50 nm to 100 nm. Particle (100) carries at least one linkage moiety (106) for releasably attaching it to a binding compound, such as an antibody, (exemplified in the figure as having a terminal biotin), and at least one leuco dye (104) represented in the figure as “LD.” Linkage moiety (106) forms a cleavable linkage between a binding compound and particle (100) Particle (100) preferably includes an indicator molecule (102)(shown as “F” in the figure) that may be attached to the surface of particle (100) or embedded in its interior, e.g. as when particle (100) is a quantum dot or Qbead (Quantum Dot Corporation, Hayward, Calif.). Indicator molecule (102) permits the degree of leuco dye conversion in an assay to bequantified ratiometrically, e.g. by a ration of a fluorescent intensity of a leuco dye conversion product to a fluorescent intensity of the indicator molecule. Preferably, indicator molecules are fluorescent molecules that are selected so that they do not interact with leuco dye (104) or its conversion product (not shown). Indicator molecules may also be used to create multiple classes of particles that are istinguishable by, for example, the optical characteristics of indicator molecules (102). Thus, multiple complexes can be measured in the same assay by using particles with different indicator molecules. In one aspect, indicator molecules (102) are fluorescent dyes having absorption bands that are substantially non-overlapping with emission bands of leuco dye conversion products.

Particle (100) records a molecular event, such as the presence of a molecular complex, in cooperation with a reactive species generator that produces a reactive species, that is, a reactive, short-lived compound, that is capable of converting a leuco dye to a fluorescent label. As illustrated in FIG. 1B, leuco dyes (104), shown as filled circles on particle (100), produce no fluorescent signal, or only minimal fluorescent signal, so that the predominant fluorescent signal form particle (100), shown in bar graph (110), is determined by indicator molecule (102), shown as gray circles on particle (100). After leuco dyes (104) are converted to fluorescent labels (108), shown as open circles on particle (100), by reaction with a reactive species, the fluorescent signal of particle (100) is changed so that it consists of both a signal from indicator molecules (102) and a signal from fluorescent labels (108), shown in bar graph (112).

An alternative form of leuco dye-labeled particle is illustrated in FIG. 1G. Here leuco dyes (170) are constructed by linking together a fluorescer (“F”) and a quencher (“Q”) by a cleavable linkage, such as a thioether linkage (“-S-”). Such conjugates are readily synthesized using commercially available materials and techniques, e.g. Haugland (cited above); Taing et al, International patent publication, WO 2002/30944; and like references. After cleavage (172) of such thioether linkage by oxidation, quenchers (176) are released and fluorescers (174) generate signals upon illumination.

In FIG. 1C, the operation of the method of the invention is illustrated for detecting a molecular dimer, such as a protein-protein dimer. Components (118 and 120) of imer (122), typically exists in equilibrium between a dimeric state and a monomeric state. In accordance with the invention, binding compound (114), shown derivatized with biotin (indicated as “bio” in the figure) specific for component (120) and reactive species generator (116) specific for are combined with a sample containing dimer (122) under conditions that permit specific binding of the reactive species generator (116) and binding compound (114) to their respective targets. For binding compounds comprising antibodies, such conditions are well-known to those of ordinary skill. e.g. Harlow and Lane (ited above). At least three different structures form (124); a complex of component (118) and reactive species generator (116); a complex of dimer (126), binding compound (114), and reactive species generator (118); a complex of binding compound (114) and component (120). To this mixture is added a multi-valent binding agent, such as avidin or strepavidin (128) that binds to the biotin on binding compound (114). After washing, isolatable particle (130) is added so that it is linked to binding compound (114) by way of the multi-valent binding agent. After isolatable particle (130) has been added reactive species generator (116), which may be a photosensitizer described more fully below, is activatd to generate a reactive species, such as singlet oxygen, which is effective within a characteristic proximity (132). Only leuco dyes within the characteristic proximity are affected by the reactive species. Leuco dyes outside of the characteristic proximity of any photosensitizer, e.g. as illustrated at (134), are not converted into fluorescent labels. After reactive species are generated and react with leuco dyes, some isolatable particles will have a portion of their leuco dyes converted into fluorescent labels, illustrated as open circles (136) in FIG. 1D. Next, the isolatable particles are released (138) from the binding compounds so that they can be separated from the reaction mixture and detected. A variety of linkage moieties are available for creating a reversible bond between an isolatable particle and a binding compound, that are well-known in the art, e.g. Hermanson, Bioconjugate techniques (Academic Press, New York, 1996); Haugland, Handbook of Fluorescent Probes and Research Products, Ninth Edition (Molecular Probes, Eugene, Oreg. 2002); and the like. In one aspect, the linkage moiety may contain a disulfide linkage, which may be cleaved using dithiothreitol (DTT) in a conventional reaction. Alternatively, isolatable particles may be derivatized with desthiobiotin for attachment to a binding compound, after which release may be effected by adding free biotin that will displace desthiobiotin from avidin or strepatavidin binding sites.

In any case, released isolatale particles are then enumerated (140), for example, by scanning with a microscope one or more fields containing such particles on a solid support, as illustrated in FIG. 1E, flow cytometric analysis, or like analytical procedure. Isolatable particles may be captured on an avidinated or strepatavidinated surface, particularly if they have been derivatized with desthiobiotin. Fields of solid support (138) may be scanned or imaged with a variety of microscope systems for detecting and enumerating particles, e.g. Stern et al, PCT publication WO 95/22058; Resnick et al, U.S. Pat. No. 4,125,828; Karnaukhov et al, U.S. Pat. No. 354,114; Trulson et al, U.S. Pat. No. 5,578,832; Bacus et al, U.S. Pat. No. 5,252,487; Bridgham et al, U.S. Pat. No. 6,406,848; and the like. In one aspect, particularly when nanometer-sizd particles are employed, a confocal optical system may be used, either in connection with a flow system transporting particles past a detection station, e.g. Ferris et al, Review of Scientific Instruments, 73: 2404-2410 (2002), which is incorporated herein by reference, or in connection with a movable microscope stage that moves particles fixed on a solid support past a detection station. An exemplary confocal scanning system is outlined diagrammatically in FIG. 1E. Illumination beam (144) generated by light source (143) passes through dichoic (145) from which it enters objective (146) and is focused on solid support (142) on which particles are located. Fluorescent signals from a particle are collected by objective (146) and are directed to dichroic (145) where they are reflected to confocal optics (147) after which the fluorescent signals are directed by mirror (148) either to filter (149) and then to photomultiplier tube (150) or to filter (151) and then to photomultiplier (152). Filters (149) and (151) are band pass filters selected to pass either fluorescence from indicator dyes or converted leuco dyes, respectively. One of ordinary skill in the art recognizes that a variety of detection schemes for comparable measurements are available using conventional optical systems, e.g. Shapiro, Practical Flow Cytometry (Wiley-Liss, New York, 1994); Murphy, Fundamentals of Light Microscopy and Electronic Imaging (Wiley-Liss, New York, 2001); Inoue and Spring, Video Microscopy—The Fundamentals (Plenum Press, New York, 1997). The readout of the above system comprises a tabulation of a bumger of particles, or a proportion of particles, that have fluorescent signal comprising substantially only fluorescence from an indicator molecule, e.g. (154), and that have a fluorescent signal comprising fluorescence from both an indicator molecule and a converted leuco dye, e.g (156). As indicated in FIG. 1E, the former corresponds to an uncomplexed, e.g. monomeric, component of a complex, and the lattercorresponds to a complexed, e.g. dimerized, component of a complex.

In one aspect, isolatable particle (130) may be a magnetic nanoparticle as illustrated in FIG. 1F, and described more fully below. The isolatable particle of FIG. 1F is the same as that shown in FIG. 1A, except that it has a core (158) made of a magnetically responsive material, described more fully below. Magnetic nanoparticles are more readily collected and washed after release from a binding compound, so that larger samples of more highly enriched particles are available for detection as described above. Such isolation before detection reduces the amount of background noise collected during a detection step.

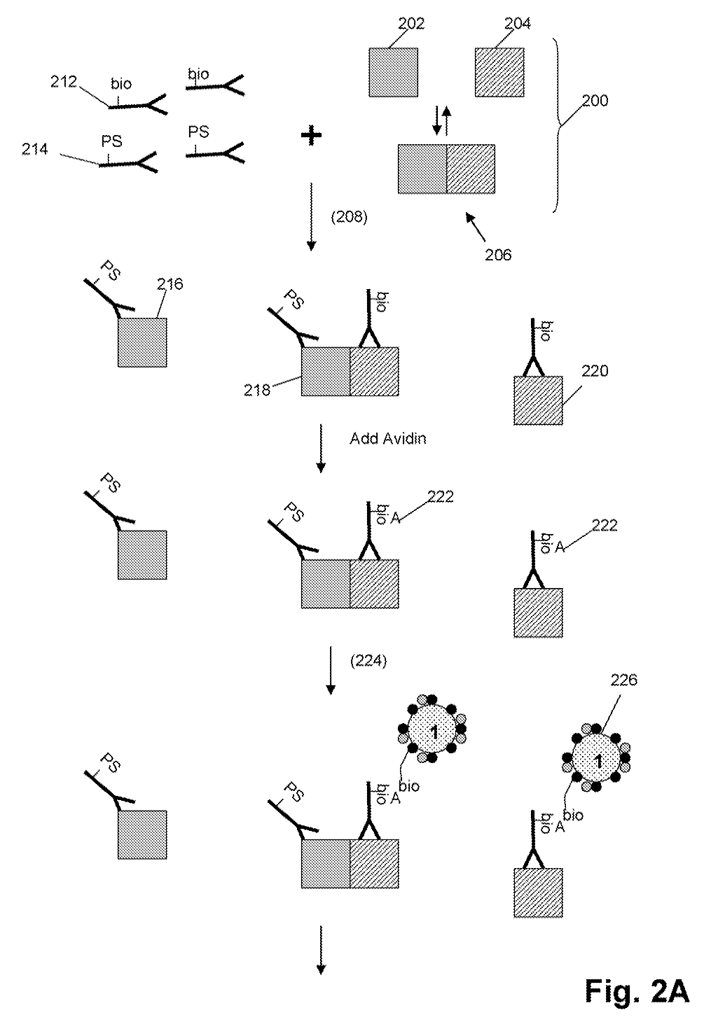

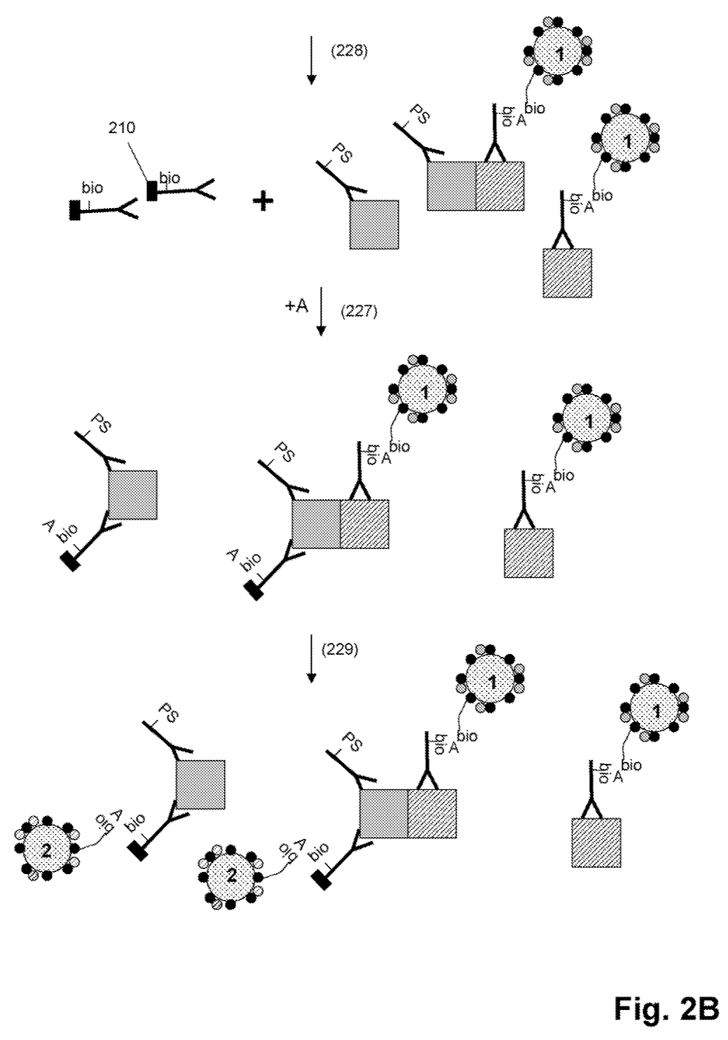

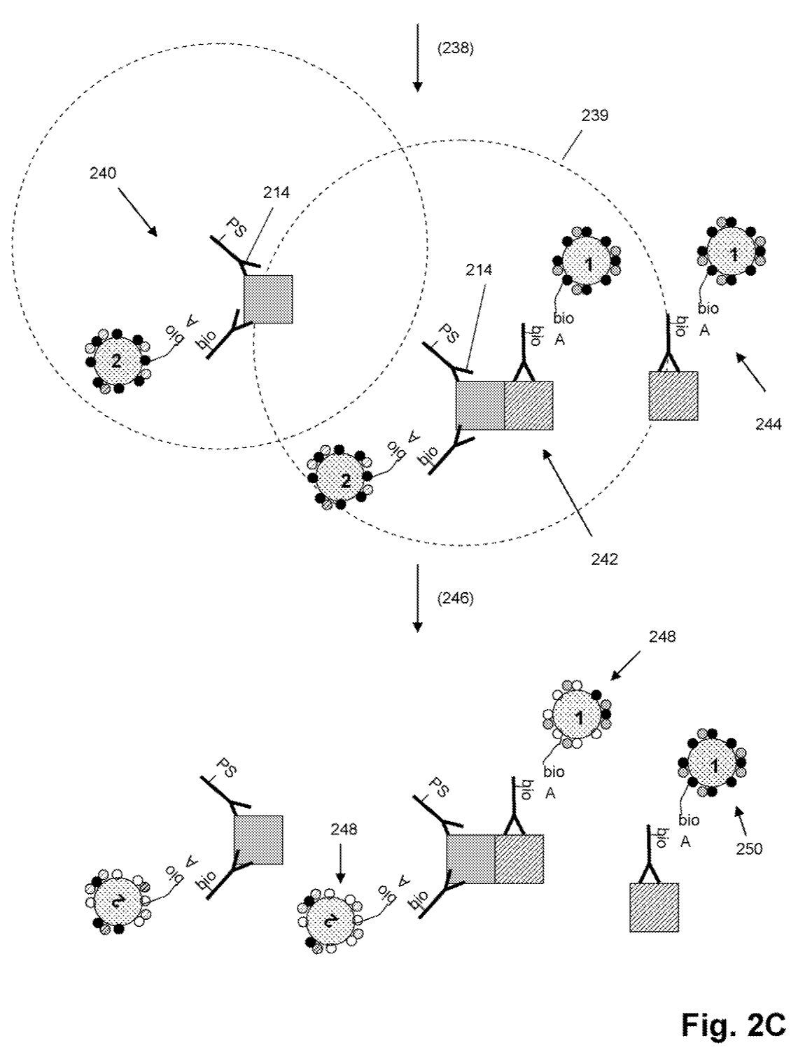

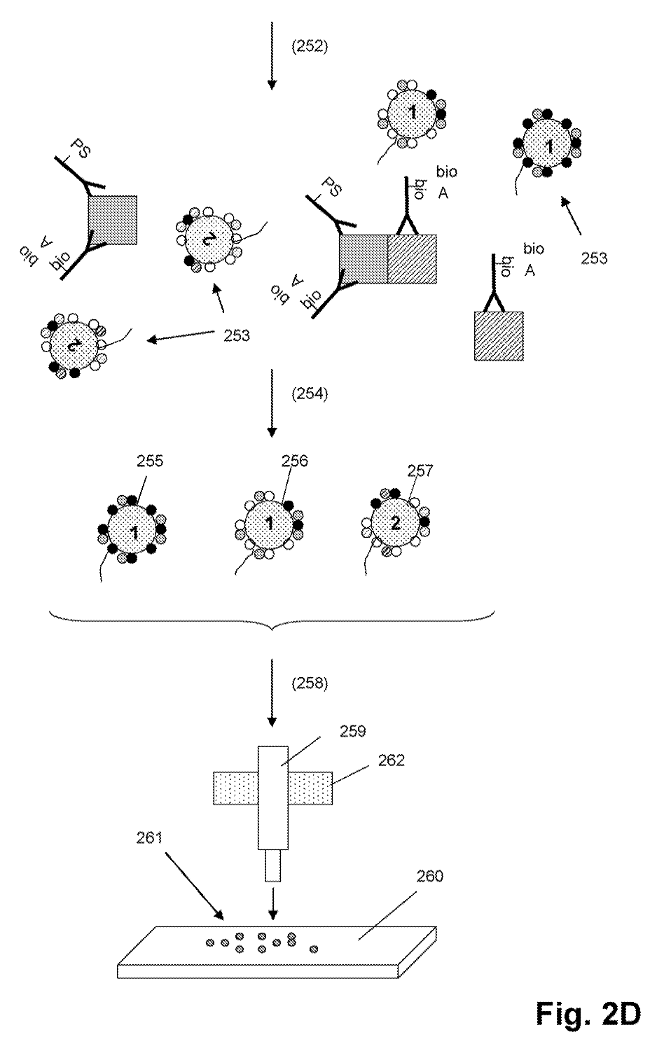

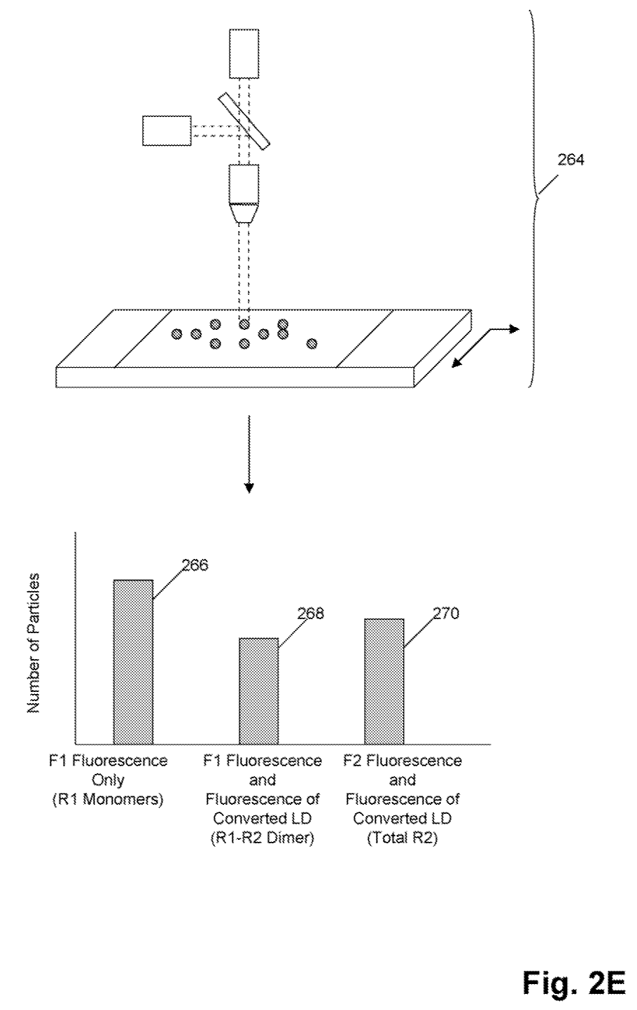

As illustrated in FIGS. 2A-2E, complexes may be detected or measured using multiple isolatable particles having distinct signal generating characteristics. In particlular, using two distinct isolatable particles, the presence and amount of dimer (206) can be given in terms of the total amount of one component present, e.g. (202), whether in dimer form or in monomer form, and the amount of the other component, e.g. (204) that is solely in dimer form. The latter quanity, of course, is the same for the first component (202); therefore, the ratio of the two quanities gives the relative amount of component (202) that is in dimer form (assuming that components (202) and (204) only form dimers with one another). This provides a measurement that is independent of variations in sample size.

An embodiment for carrying out such measurements is illustrated in FIGS. 2A-2E. Binding compound (212) and cleaving probe (214) are provide that are specific for components (202) and (204), respectively. Binding compounds (212) and cleaving probe (214) are combined (208) with a sample containing dimer (206) under conditions that promote the specific binding of cleaving probe (214) to component (202) and specific binding of binding compound (212) to component (204). Since components (202) and (204) may exist as a dimer (206) or as monomers (which may be an equilibrium condition (200) or an actively maintained state in a biological system), some of binding compound (212) will be bound to monomers (220), some of cleaving probe (214) will be bound to monomers (216), and some of both will be bound to dimers (218). Avidin or streptavidin is added (221) which binds to the biotinylated binding compounds (222). After removal of excess avidin or streptavidin, biotinylated isolatable particles (226, designated by “2”) are added (224). To this mixture, binding compounds (210) are added (228) so that they specifically bind to component (202) at an epitope separate from that to which cleaving probe (214) binds, after which avidin or streptavidin is again added (227). After removal of excess avidin or streptaviding, a second isolatable particle is a added (229, designated by 2). Alternatively, instead of such successive additions of isolatable particles, by using different hapten-capture agent combinations, both particles can be added in the same step. Once the two isolatable particles have speciically bound to their target epitomes, and appropriate washing, the reaction mixture is illuminated (238) to cause the photosensitizers of cleaving probes (214) to generated singlet oxygen within an effective proximity (239). As noted above, within the reaction mixture, because the components (202 and 204) may exist in either monomeric form or dimeric form, at least three configurations are possible: (240) where both cleaving probe (214) and binding compound (210) are bound to monomeric (202), (242) where cleaving probe (214) and binding compounds (210) and (212) are bound to a dimer, and (244) where binding compound (212) alone is bound to monomeric (204). Thus, only the subset of isolatable particles within the effective procimities of cleaving probes (248) will have their leuco dyes converted into fluorescent signal generating labels (illustrated by the unfilled circles in FIG. 2C). Leuco dyes of isolatable particles (250) outside any effective proximity will remain non-fluorescent. After illumination (252) to activate photosensitizers on cleaving probes (214), particles are released (253) and isolated (254) to give three populations: particles with inactivated leuco dyes (255), particles with activated (and therefore fluorescent) irst leuco dyes (256), and activated second leuco dyes (257). In one aspect, these populations are isolated magnetically (258) using a conventional column (259) and magnet (262) for separating magnetic nanoparticles, e.g. Miltenyi Biotec (Cologne, Germany). the separated particles (261) are then disposed on a surface (260) where particles in the three populations are enumerated using, for example, a confocal microscope (264). Results may be displayed in graphical form with bars (266), (268), and (270) showing the relative amounts of each of the particle types.

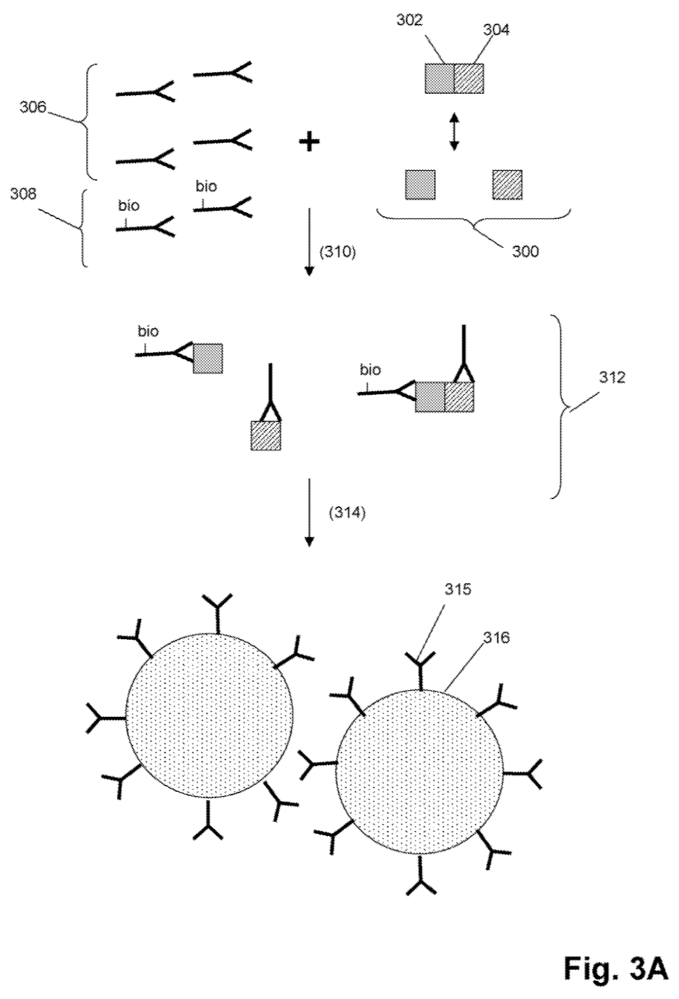

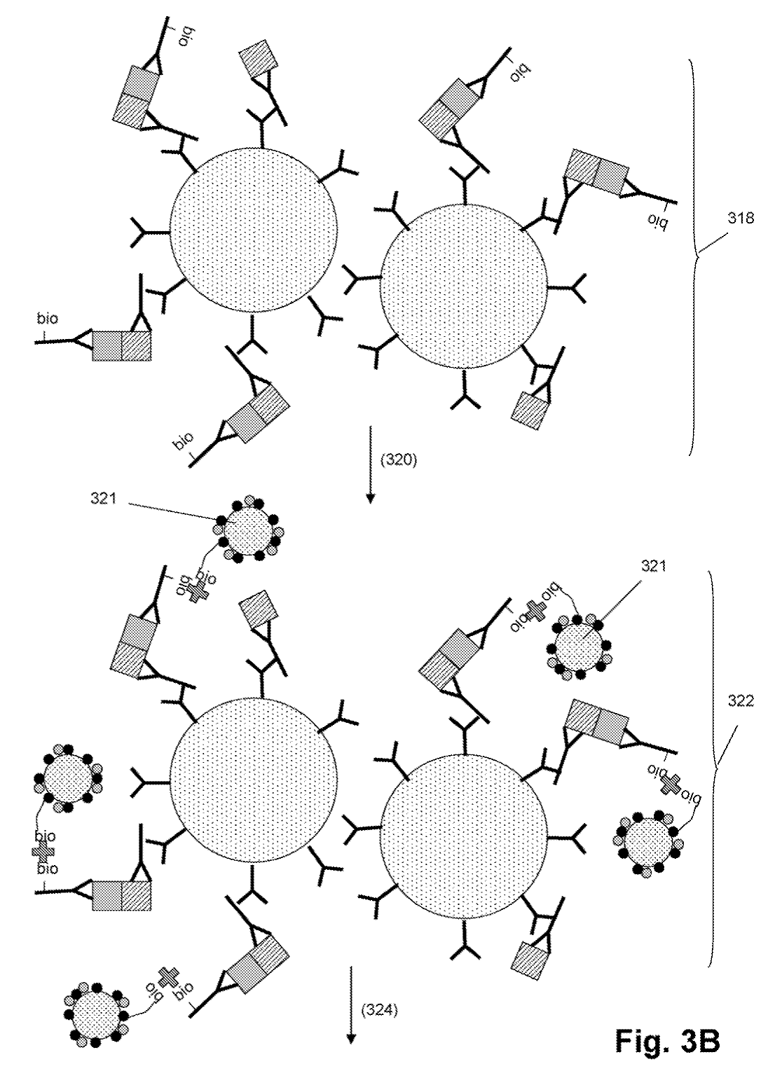

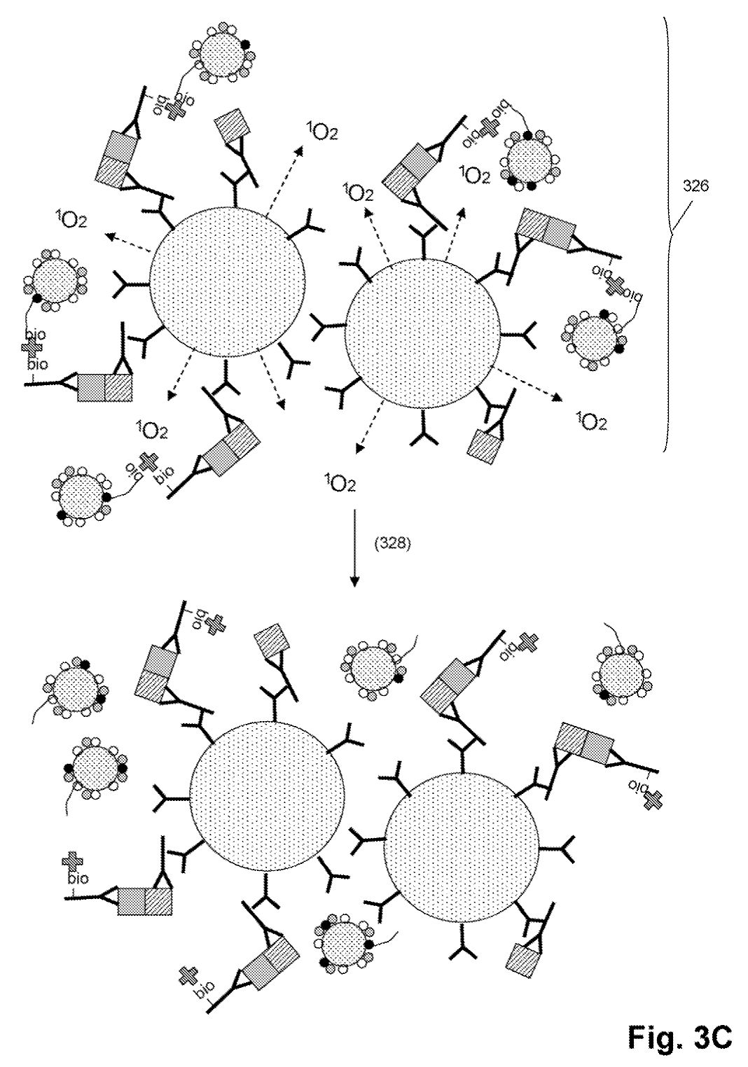

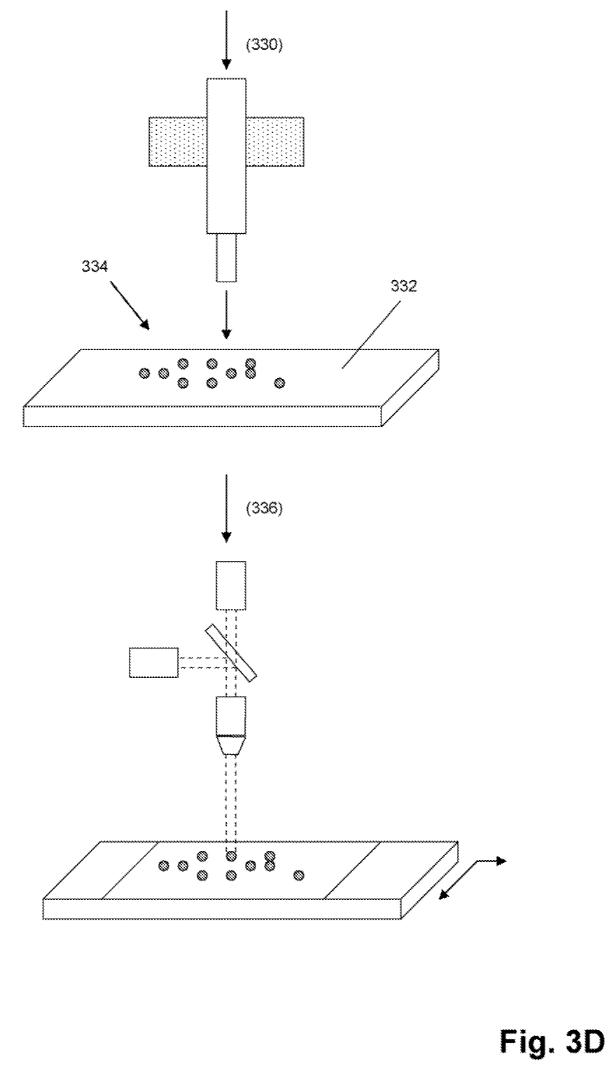

Another embodiment for detecting molecular complexes that employes photo sensitizer beads is illustrated in FIGS. 3A-3D. Molecular complex (300) in equilibrium between a complexed state of components (302) and (304) and a dissociated state are combined with antibodies (360) specific for component (304) and biotinylated antibodies (308) specific for component (302) so that three different types of complexes form (312). Complexes (312) are then combined with photosensitizer particles (316) derivizatized with antibody (315) specific for the isotype of antibody (306) resulting in particle-bound complexes (318). To these complexes leuco dye-labeled particles (321) are attached (320) to viotinylated antibodies (308) through a streptavidin or avidin bridge, as illustrated (322). These complexes are illuminated (324) to stimulate particles (316) to generate a reactive species, such as singlet oxygen, thereby activating leuco dyes within the effective proximity of particles (316). Leuco dye-labeled particles (321) are then released (328), isolated (330), and disposed (334) on surface (332) for detection and enumeration (336), as described above.

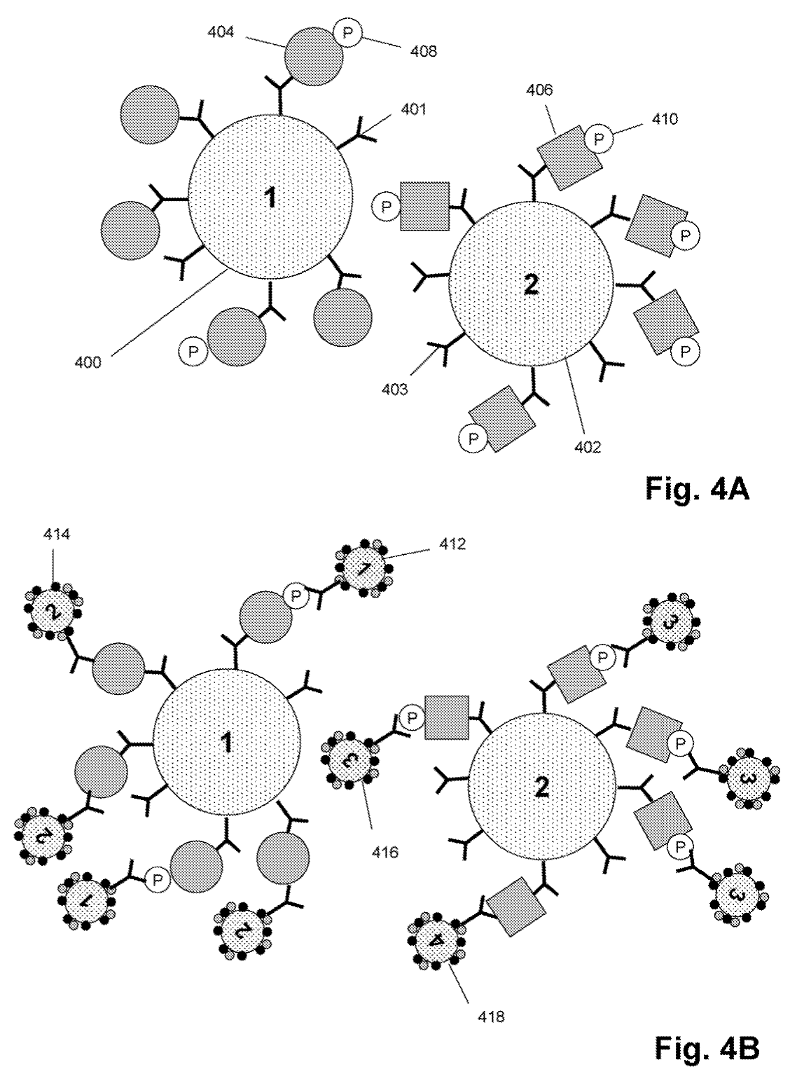

In another aspect, the invention provides a method of measuring relative phosphorylation states of one or more proteins, for example, as illustrated in FIGS. 4A and 4B. Photosensitizer beads (400) and (402) are provided that have antibodies (401) ad (403) attached that are specific for phosphoprotein 1 (404) and Phosphoprotein 2 (406). In a sample, phosphoprotein 1 may or may not be phosphorylated at a particular site (408) Likewise, phosphoprotein 2 may or may not be phosphorylated at a particular site (410). Composition of the invention comprising four different particles (412-418), e.g. nanoparticles, are combined with photosensitizer beads 1 and 2 so that the respective antibodies specifically bind to their targets. Each different particle has attached a different indicator molecule, or each such particle can be a bead having distinct spectral characteristics, e.g. it can be a distinct quantum dot or the like. After activation of the photosensitizer beads, the leucodyes on the particles are converted to signal generating moieties and can be detected as described above. In this embodiment, a releasable linkage to the binding moiety is optional. Alternatively, the particles may be simply be dissociated from the complex and separated for measurement, or in some embodiments, measurement of the changes in optical signals (e.g. indicator fluorescence versus signal generating moiety fluorescence) before and after activation of the photosensitizer beads can be carried out without dissociation or deparation.

### Sample Preparation

Samples containing molecular complexes may come from a wide variety of sources including cell cultures, animal or plant tissues, microorganisms, patient biopsies, or the like. Samples are prepared for assays of the invention using conventional techniques, which may depend on the source from which a sample is taken. Guidance for sample preparation techniques can be found in standard treatises, such as Sambrook et al, Molecular Cloning, Second Edition (Cold Spring Harbor Laboratory Press, New York, 1989): Innis et al, editors, PCR Protocols (Academic Press, New York, 1990); Berger and Kimmel, “Guide to Molecular Cloning Techniques,” Vol. 152, Methods in Enzymology (Academic Press, New York, 1987); Ohlendieck, K. (1996). Protein Purification Protocols; Methods in Molecular Biology, Humana Press Inc., Totowa, N.J. Vol 59: 293-304; Method Booklet 5, “Signal Transduction” (Biosource International, Camarillo, Calif. 2002); or the like.

For blood specimens, the following references provide guidance for separating red blood cells from other cells in a specimen and for combining such other cells with immunomagnetic particles: Nakamura et al, Biotechnol. Prog., 17: 1145-1155 (2001); Moreno et al, Urology, 58: 386-392 (2001); Racila et al. Proc. Natl. Acad. Sci., 95: 4589-4594 (1998); Zigeuner et al, J. Urology, 169: 701-705 (2003); Ghossein et al, Seminars in Surgical Oncology, 20: 304-311 (2001): Terstappen et al, U.S. Pat. No. 6,365,362.

For biopsies and medical specimens, guidance for sample preparation is provided in the following reerences: Bancroft J D & Stevens A, eds. Theory and Practice of Histological Techniques (Churchill Livingstone, Edinburgh, 1077): Pearse, Histochemistry. Theory and applied. 4^{th} ed. (Churchill Livingstone, Edinburgh, 1980).

Samples are prepared for assays of the invention using conventional techniques, which may depend on the sourde from which a sample is taken.

Examples of patient tissue samples that may be used include, but are not limited to, breast, prostate, ovary, colon, hung, endothelium, stomach, salivary gland or pancreas. A tissue sample can be obtained by a variey of procedurs including, but not limited to surgical excision, aspiration or biopsy. The tissue may be fresh or frozen. In one embodiment, assays of the invention are carried out on tissue samples that have been fixed and embedded in paraffin or the like; therefore, in such embodiments a step of deparafination is carried out.

For mammalian tissue culture cells, fresh or frozen tissue specimens, or like sources, samples of complexes may be prepared by conventional cell lysis techniques (e.g. 0.14 M NaCl, 1.5 mM MgCl, 10 mM Tris-Cl (pH 8.6), 0.5% Nonidet P-40, and protease and/or phosphatase inhibitors as required).

### Particles

A wide variety of particle and microparticle supports may be used with the invention, including microparticles made of controlled pore glass (CPG), highly cross-linked polystyrene, acrylic copolymers, cellulose, nylon, dextran, latex, polyacrolein, and the like, discloled in the following exemplary references: Meth. Enzymol., Section A, pages 11-147, vol. 44 (Academic Press, New York, 1976); U.S. Pat. Nos. 4,678,814; 4,413,070; and 4,046,720; Pon, Chapter 19, in Agrawal, editor, Methods in Molecular Biology, Vol. 20, (Humana Press, Totowa, N.J., 1993); TechNote 205, Rev. 003, Mar. 30, 2002 (Bangs Laboratores, Fishers, Ind.). Microparticle supports further include commercially available nucleoside-derivatized CPG and polystyrene beads (e.g. available from Applied Biosystems, Foster City, Calif.); derivatized magnetic beads; polystyrene grafted with polyethylene glycol (e.g., TentaGel. Tm., Rapp Polymere, Tubingen Germany); and the like. Exemplary linking moieties for attaching molecules on microparticle surfaces are disclosed in Hermanson, bioconjugate Techniques (Academic Press, New York, 1996); Pon et al, Biotechniques, 6:768-775 (1988); Webb, U.S. Pat. No. 4,659,774; Barany et al, International patent application PCT/US91/06103; Brown et al, J. Chem. Soc. Commun., 1989; 891-893; Damha et al, Nucleic Acids Research, 18: 3813-3821 (1990); Beattie et al, Clinical Chemistry, 39:719-722 (1993); Maskos and Southern, Nucleic Acids Research, 20: 1679-1684 (1992); and like references.

In one aspect, particles used in signaling reagents are nanoparticles, and in particular magnetic nanoparticles. A wide variety of techniques and materials employing magnetic particles for biomolecular assays are well known in the art, as disclosed in the following representative references that are incorporated by reference: Terstappen et al, U.S. Pat. No. 6,365,362; Terstappen et al, U.S. Pat. No. 5,646,001; Rohr et al, U.S. Pat. No. 5,998,225; Kausch et al, U.S. Pat. No. 5,665,582; Kresse et al, U.S. Pat. No. 6,048,515; Kausch et al, U.S. Pat. No. 5,508,164: Miltenyi et al, U.S. Pat. No. 5,691,208;Molday, U.S. Pat. No. 4,452,773; Kronick, U.S. Pat. No. 4,375,407; Radbruch et al, chapter 23, in Methods in Cell Biology, Vol. 42 (Academic Pres, New York, 1994); Uhlen et al, Advances in Biomagnetic Separation (Eaton Publishing, Natick, 1994); Safarik et al, J. Chromatography b, 722: 33-53 (1999); Miltenyi et al, Cytometry, 11;231-238 (1990); Nakamura et al, Biotechnol. Prog., 17: 1145-1155 (2001); Moreno et al, Urology, 58: 386-392 (2001); Racila et al, Proc. Natl. Acad. Sci., 95: 4589-4594 (1998); Zigeuner et al, J. Urology, 169: 701-705 (2003); Ghossein et al, Seminars in Surgical Oncology, 20: 304-311 (2001); and U.S. Pat. Nos. 4,795,698, 5,597,531 and 5,698,271.

### Releasable Linkages

In one aspect, commercially available cleavable reagent systems may be employed with the inventon. For example, a disulfide linkage may be introduced between an antibody binding composition and a molecular tag using a heterofunctional agent such as N-succinimidyl 3-(2-pyridyldithio)propionate (SPDP), succinimidyloxycarbonyl-α-(2-pyridyldithio)toluene (SMPT), or the like, available from vendors such as Pierce chemical Company (Rockford, Ill.). Disulfide bonds introduced by such linkages can be broken by treatment with a reducing agent, such as dithiothreitol (DTT), dithioerythritol (DTE), 2-mercaptoethanol, sodium borohydride, or the like. Typical concentrations of reducing agents to effect cleavage of disulfide bonds are in the range of from 10 to 100 mM. An oxidatively lavile linkage may be introduced between an antibody binding composition and a molecular tag using the homobifunctional NHS ester cross-linking reagent, disuccinimidyl tartarate (DST)(available from Pierce) that contains central cis-diols that are susceptible to cleavage with sodium periodate (e.g., 15 mM periodate at physiological pH for 4 hours). Linkages that contain esterified spacer components may be cleaved with strong nucleophilic agents, such as hydroxylamine, e.g. 0.1 N hydroxylamine, pH 8.5, for 3-6 hours at 37° C. Such spacers can be introduced by a homobifunctional cross-linking agent such as ethylene glycol bis(succinimidylsuccinate)(EGS) available from Pierce (Rockford, Ill.). A baase labile linkage can be introduced with a sulfone group. Homobifunctional cross-linking agents that can be used to introduce sulfone group in cleavable linkage include bis[2-(succinimidyloxycarbonyloxy)ethyl]sulfone (BSOCOES, and 4,4-difluoro-3,3-dinitrophenylsulfone (DFDNPS). Exemplary basic conditions for cleavage include 0.1 M sodium phosphate, adjusted to pH 11.6 by addition of Tris Base, containing 6 M urea, 0.1% SDS, and 2 mM DTT, with incubation at 37° C. for 2 hours. Photocleavable linkages include those disclosed in Rothschild et al, U.S. Pat. No. 5,986,076.

### Leuco Dyes and Fluorescent Labels

Leuco dyes and fluorescent label that can be used in connection with the invention are present invention. Such a reagent is normally present at concentrations as discussed below. The photosensitizer excited state usually has a different spin quantum number S than its ground state and is usually a triplet (S=1) when the ground state, as is usually the case, is a singlet (S=0). Preferably, the photosensitizer has a high intersystem crossing yield. That is, photoexcitation of a photosensitizer usually produces a triplet state with an efficiency of at least about 10%, desirably at least about 40%, preferably greater than about 80%.

Photosensitizers chosen are relatively photostable and, preferably, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they may have extended aromatic structures.

A large variety of light sources are available to photo-activate photosensitizers to generate singlet oxygen. Both polychromatic and monochromatic sources may be used as long as the source is sufficiently intense to produce enough singlet oxygen in a practical time duration. The length of the irradiation is dependent on the nature of the photosensitizer, the nature of the cleavable linkage, the power of the source of irradiation, and its distance from the sample, and so forth. In general, the period for irradiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and not limitation lasers such as e.g., helium-neon lasers, argon lasers, YAG lasers, He/Cd lasers, and ruby lasers; photodiodes; mercury, sodium and xenon vapor lamps; incandescent lamps such as e.g., tungsten and tungsten/halogen; flashlamps; and the like. An exemplary photodiode for stimulating photosensitizers to generate singlet oxygen is a high power GaAlAs IR emitter LED, such as model OD-880W laser diode manufactured by OPTO DIODE CORP. (Newbury Park, Calif.).

Examples of photosensitizers that may be utilized in the present invention are those that have the above properties and are enumerated in the folloeing references: Singh and ullman, U.S. Pat. No. 5,536,834; Li et al, U.S. Pat. No. 5,763,602; Martin et al, Methods Enzymol., 186: 635-645 (1990); Yarmush et al. Crit. Rev. Therapeutic Drug Carrier Syst., 10: 197-252 (1993); Pease et al, U.S. Pat. No. 5,709,994; McCapra, U.S. Pat. No. 5,516,636; Thetford, European patent publ. 0484027; Sessler et al, SPIE, 1426: 318-329 (1991); Magda et al, U.S. Pat. No. 5,565,552; Roelant, U.S. Pat. No. 6,001,673; and the like.

As with sensitizers, in certain embodiments, a photosensitizer may be associated with a solid phase support by being covalently or non-covalently attached to the surface of the support or incorporated into the body of the support. In general, the photosensitizer is associated with the support in an amount necessary to achieve the necessary amount of singlet oxygen. Generally, the amount of photosensitizer is determined empirically. In one preferred embodiment, a photosensitizer is incorporated into a latex particle to form photosensitizer beads, e.g. as disclosed by Pease et al., U.S. Pat. No. 5,709,994; Pollner, U.S. Pat. No. 6,346,384; Patel, PCT publication WO 01/90399; and Pease et al, PCT publication WO 01/84157. In another exemple the photosensitizer Rose Bengal is covalently attached to 0.5 micron latex beads by means of chloromethyl groups on the latex to provide an ester linking , as described in J. Amer. Chem. Soc,m 97: 3741(1975).

In another aspect, a reactive species generator may comprise an avidin or streptavidin derivatized with photosensitizer molecules, such as methylene blue, that is conjugated to a biotinylated antibody, such as disclosed in International patent publication W) 2005/037071.

### Exemplary Molecular Complexes

Exemplary molecular complexes that may be analyzed with methods of the invention include those listed below.

TABLE I
Exemplary Receptor Tyrosine Kinase Dimers and Intracellular
Complexes (here “protein 1//protein 2” indicates a
complex comprising protein 1 and protein 2)

| RTK Dimer | Downstream Complexes |
|---|---|
| Her1-Her1 | Her1//She, Grb2//Sos, Her1//Grb7, Her1//RasGAP |
| Her1-Her2 | Her1//She, Grb2//She, Her2//She, Grb2//Sos, 14-3-3//Bad, |
| | Her1//RasGAP |
| Her1-Her3 | Her3//PI3K, Her3//She, Her3//Grb7, Her1//She, Grb2//Sos, 14-3-3//Bad, |
| | Her1//RasGAP |
| Her1-Her4 | Her3//PI3K, Her1//She, Grb2//Sos, Her1//RasGAP |
| Her2-Her2 | Her2//She, Grb2//Sos, 14-3-3//Bad, Her1//RasGAP |
| Her2-Her3 | Her3//PI3K, Her3//She, Her3//Grb7, Grb2//She, Her2//She, Grb2//Sos, |
| | 14-3-3//Bad, Her1//RasGAP |
| Her2-Her4 | Her3//PI3K, Grb2//She, Her2//She, Grb2//Sos, 14-3-3//Bad; YAP//Her4, |
| | Her1//RasGAP |
| Her3-Her4 | Her3//PI3K, Her3//She, Her3//Grb7, YAP//Her4, Her1//RasGAP |
| Her4-Her4 | Her3//PI3K, YAP//Her4, Her1//RasGAP |
| VEGFR1(Flt1)- | VEGFR//She; VEGFR//PI(3)K; VEGFR//Src; VEGFR//FRS2 |
| VEGFR2(KDR) | |
| VEGFR2(KDR)- | VEGFR//She; VEGFR//PI(3)K; VEGFR//Src; VEGFR//FRS2 |
| VEGFR2(KDR) | |
| PDGFRa-PDGFRa | PDGFRa//Crk, PDGFR//Grb2; PDGFR//Grb7; PDGFR//Nck; |
| | PDGFR//She; PDGFR//STAT5 |
| PDGFRa-PDGFRb | PDGFRa//Crk; PDGFRb//GAP, PDGFR//Grb2; PDGFR//Grb7; |
| | PDGFR//Nck; PDGFR//She, PDGFR//Shp2; PDGFR//RasGAP, |
| | PDGFR//STAT5 |
| PDGFRb-PDGFRb | PDGFRb//GAP, PDGFR//Grb2, PDGFR//Grb7; PDGFR//Nck; |
| | PDGFR//She, PDGFR//Shp2, PDGFR//RasGAP; PDGFR//STAT5 |

TABLE II
Exemplary Receptor Complexes of Cell Surface Membranes

| Dimer | Dimer |
|---|---|
| Her1-Her1 | IGF-1R-Her1 heterodimer |
| Her1-Her2 | IGF-1R-Her3 heterodimer |
| Her1-Her3 | Her1-PDGFR heterodimers |
| Her1-Her4 | Her3-PDGFR heterodimers |
| Her2-Her2 | Her2-PI3K |
| Her2-Her3 | Her1-SHC |
| Her2-Her4 | Her3-SHC |
| Her3-Her4 | Her2-SHC |
| Her4-Her4 | Her3-PI3K |
| Her2-PDGFR heterodimers | Her1-PI3K |
| IGF-1R-Her2 heterodimer | |

### EXAMPLE

### Magnetic Nanoparticle Derivatized with Desbiotin Dichlorodihydrofluorescein and Alexa 633

In this example, magnetic nanoparticles derivatized with dextran as taught by Rudershausen et al (European Cells and Materials. Vol. 3, Suppl. 2, pgs. 81-83 (20002)) are reacted with equimolar amounts of NHS-esters of dichlorodihydrofluorescein, Alexa 633, and desbiotin. After puriication, the nanoparticles are incubated in solutions of different concentrations of methylene blue (as photosensitizer) that are each irradiated with the same diode laser for diferent amounts of time. The magnetic nanoparticles are then isolated by a MACS Separator (Miltenyi Biotec, Cologne, Germany) and deposited on an avidinated microscope slide (e.g., SAM biotin capture membrane, Promega, or the like) and examined with a confocal microscope, e.g. using a system as disclosed in Ferris et al. Review of Scientific Instruments, 73: 2404-2410 (2002), or the like, to count particles and measure fluorescence ratios on each particle.

What is claimed is:

1. A particle composition for detecting molecular complexes, the composition comprising:
a plurality of isolatable particles having alargest dimension of less than or equal to about 300 nanometers and each having a surface;
leuco dyes attached to the surface of the isolatable particle, the leuco dyes capable of being converted into detection moieties by a reactive species; and
a binding compound specific for a molecular complex releasably attached to the surface of each isolatable particle such that after the leuco dyes are converted in th presence of the molecular complex ino detection moieties capable of generating an optical signal.

2. The particle composition of claim 1 wherein each of said isolatable particles further comprises an indicator compound that generates a second optical signal having different optical characteristics than those of said optical signal o said detection moiety, so that an amount of said detection moiety is measured on said isolatable particle by comparison of said optical signal of said detection moiety to the second optical signal of the indicator compound.

3. A composition comprising a plurality of nanoparticles each havin a surface and each having attached to such surface (i) leuco dyes capable of being converted into detection moieties by a reactive species, the detection moieties being capable of generating a first optical signal and (ii) one or more binding compounds each having a specificity fo an antigen, such that the specificity of the one or more binding compounds on the same nanoparticle is for the same antigen, and such that the specificity of the one or more binding compounds on each different nanoparticle of the plurality is for a different antigen.

4. The composition of claim 3 wherein each of said nanoparticles further comprises an indicator compound capable of generating a second optical signal.

5. The composition of claim 4 wherein said nanoparticles are isolatable nanoparticles and wherein said one or more binding compounds of each of said isolatable nanoparticles are releasably attached to said surface of said isolatable nanoparticle.

6. The composition of claim 5 wherein each said different antigen is a different protein in a molecular complex.

7. A composition comprising:
(a) an isolatable nanoparticle having attached to its surface (i) leuco dyes capable of being converted into detection moieties by a reactive species, and (ii) one or more binding compounds specific for a molecular complex, the one or more binding compounds being releasably attached to the surface of the isolatable nanoparticle; and
(b) a cleaving probe specific for the molecular complex, the cleaving probe having a reactive species generator for generating a reactive species for converting the leuco dyes into detection moieties.
